(19) United States
(12) Patent Application Publication
(10) Pub. No.: US 2020/0163590 A1
(43) Pub. Date: May 28, 2020

(54) FALL DETECTION METHOD, DEVICE, AND SYSTEM

(71) Applicant: JOMOO KITCHEN & BATH CO., LTD, Nan'an City (CN)

(72) Inventors: Xiaofa LIN, Nan'an City (CN); Xiaoshan LIN, Nan'an City (CN); Jinyu HU, Nan'an City (CN)

(73) Assignee: JOMOO KITCHEN & BATH CO., LTD, Nan'an City (CN)

(21) Appl. No.: 16/591,991

(22) Filed: Oct. 3, 2019

(30) Foreign Application Priority Data
Nov. 22, 2018 (CN) 201811399469.1

(51) Int. Cl. A61B 5/11; A61B 5/00; H04B 17/30; G06N 3/04; G06N 3/08

(52) U.S. Cl. CPC A61B 5/1117; A61B 5/1126; A61B 5/7203; A61B 5/7253; G06N 3/08; H04B 17/30; G06N 3/04; G06N 3/0454; A61B 5/7264

(57) ABSTRACT

A fall detection method, device, and system are disclosed in this application for detecting whether a target object falls in a detection area. The fall detection method includes: receiving a WIFI signal transmitted by a transmitter in the detection area and extracting CSI data from the WIFI signal; preprocessing the CSI data to obtain CSI data to be identified, and processing the CSI data to be identified through a deep neural network to determine whether the target object falls in the detection area. In this application, a deep neural network is adopted to perform fall detection and the detection accuracy is improved.

### CROSS-REFERENCE TO RELATED APPLICATIONS

This application claims foreign priority benefits under 35 U.S.C. § 119(a)-(d) to Chinese patent application number CN 201811399469.1, filed on Nov. 22, 2018, which is incorporated by reference in its entirety.

### TECHNICAL FIELD

This application relates to, but not limited to, the field of computer technology, and in particular, to a fall detection method, device and system.

### BACKGROUND

Falling has become a major cause of fatal and non-fatal injuries of the elderly in modem society. Currently, fall detection may be performed based on Channel State Information (CSI) of WIFI signals. Fall recognition may be carried out in the following two ways: histogram-based and machine learning-based. When fall recognition is performed based on a histogram, the histogram of CSI may be compared with a database to find nearest CSI, so as to identify the fall activity of a human body. However, a histogram is very sensitive to an environmental change, and after an environment change is detected, the effect of detection by a histogram is not good. When fall recognition is performed based on machine learning, for example, logistic regression, Support Vector Machine (SVM), Hidden Markov Model and so on may be used. However, traditional machine learning methods are greatly influenced by the environment, and it is difficult to distinguish similar activities (such as sitting or lying down), resulting in a low accuracy of detection results.

### SUMMARY

Embodiments of this application provide a fall detection method, device and system, in which a deep neural network is adopted to perform fall detection and the detection accuracy is improved.

In one aspect, an embodiment of this application provides a fall detection method for detecting whether a target object falls in a detection area. The fall detection method includes receiving a WIFI signal transmitted by a transmitter in the detection area and extracting channel state information (CSI) data from the WIFI signal; preprocessing the CSI data to obtain CSI data to be identified; and processing the CSI data to be identified through a deep neural network to determine whether the target object falls in the detection area.

In another aspect, an embodiment of this application provides a fall detection device for detecting whether a target object falls in a detection area. The fall detection device includes: a receiving module, adapted to receive a WIFI signal transmitted by a transmitter in the detection area, and extract CSI data from the WIFI signal; a preprocessing module, adapted to preprocess the CSI data to obtain CSI data to be identified; and a deep neural network, adapted to process the CSI data to be identified to determine whether the target object falls in the detection area.

In yet another aspect, an embodiment of this application provides a terminal including a receiver, a memory and a processor. The receiver is connected to the processor and is adapted to receive a WIFI signal transmitted by a transmitter in a detection area, and the memory is adapted to store a fall detection program, which, when executed by the processor, realizes the steps of the fall detection method mentioned above.

In yet another aspect, an embodiment of this application provides a fall detection system for detecting whether a target object falls in a detection area. The fall detection system includes a transmitter and a data processing terminal. The transmitter is adapted to transmit a WIFI signal in the detection area. The data processing terminal is adapted to receive the WIFI signal transmitted by the transmitter in the detection area and extract CSI data from the WIFI signal; preprocess the CSI data to obtain CSI data to be identified; and process the CSI data to be identified through a deep neural network to determine whether the target object falls in the detection area.

In yet another aspect, an embodiment of this application provides a computer readable medium in which a fall detection program is stored. The fall detection program, when executed by the processor, realizes the steps of the fall detection method mentioned above.

In the embodiments of this application, CSI data are extracted from a WIFI signal and CSI data to be identified are processed through a deep neural network to identify whether a target object falls in a detection area, thus improving the accuracy of detection results.

Other characteristics and advantages of this application will be described in the following contents of the specification, and, in part, become apparent from the specification or are understood by implementing this application. The purpose and other advantages of this application may be realized and obtained by the structure specifically indicated in the specification, the claims and the accompanying drawings.

### BRIEF DESCRIPTION OF THE DRAWINGS

The accompanying drawings are used to provide further understanding of a technical solution of the application, form a part of the specification, and are used together with embodiments of the application to explain the technical solution of the application, but do not constitute a limitation on the technical solution of the application.

FIG. 1 is a flowchart of a fall detection method provided by an embodiment of this application;

FIG. 2 is a schematic diagram of a fall detection device provided by an embodiment of this application;

FIG. 3 is a schematic diagram of an application example provided by an embodiment of this application;

FIG. 4 is a schematic diagram of a process of extracting CSI amplitude data to be identified from a spectrum diagram in the above application example;

FIG. 5 is a schematic diagram of the construction of a deep neural network in an embodiment of this application;

FIG. 6 is a schematic diagram of three data collection environments in an embodiment of this application;

FIG. 7 is an exemplary diagram of fall and fall-like in an embodiment of this application;

FIG. 8 is a schematic diagram of a terminal provided by an embodiment of this application; and

FIG. 9 is a schematic diagram of a fall detection system provided by an embodiment of this application.

### DETAILED DESCRIPTION

Details of embodiments of this application are described in detail below in conjunction with the accompanying drawings. It should be noted that, without conflict, embodiments in this application and characteristics in the embodiments may be arbitrarily combined with each other.

The steps illustrated in the flowchart may be performed in a computer system such as a set of computer-executable instructions. Although a logical order is shown in the flowchart, in some cases, the steps shown or described may be performed in an order different from here.

Embodiments of this application provide a fall detection method, device and system for detecting whether a target object falls within a detection area. Target objects may include movable objects such as a human body, an animal body, etc., and detection areas may include indoor environments such as a bedroom, a bathroom, a toilet, etc. However, this application is not limited thereto.

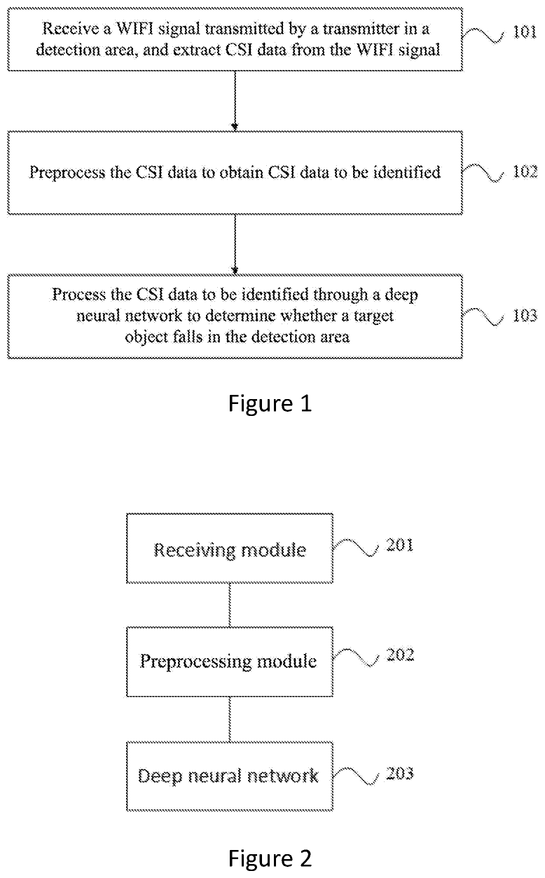

FIG. 1 is a flowchart of a fall detection method provided by an embodiment of this application. The fall detection method provided in this embodiment may be performed by a terminal (for example, a mobile terminal such as a notebook computer, or a personal computer, or a fixed terminal such as a desktop computer). In an exemplary embodiment, a transmitter and the terminal may be configured within a detection area. The transmitter is adapted to transmit a WIFI signal, and the terminal may receive the WIFI signal transmitted by the transmitter in the detection area, and conduct fall detection based on the received WIFI signal.

As shown in the FIG. 1, the fall detection method provided by this embodiment includes the following steps 101-103.

In Step 101, a WIFI signal transmitted by a transmitter in a detection area is received, and CSI data are extracted from the WIFI signal.

In Step 102, the CSI data are preprocessed to obtain CSI data to be identified.

In Step 103, the CSI data to be identified are processed through a deep neural network to determine whether a target object falls in the detection area.

In an exemplary embodiment, CSI data may include CSI amplitude data. However, this application is not limited thereto. In other implementations, CSI data may include CSI phase difference data. Compared with the CSI phase difference data, by using the CSI amplitude data for fall detection, the training efficiency of the deep neural network can be improved and the training time of the deep neural network can be avoided from being too long.

In an exemplary embodiment, Step 102 may include: using a Singular spectrum Analysis (SSA) algorithm for denoising the CSI amplitude data; converting the denoised CSI amplitude data into a spectrum diagram through Hilbert-Huang Transform (HHT); and extracting CSI amplitude data of fall or fall-like from the spectrum diagram to be used as the CSI data to be identified.

In this exemplary embodiment, after the CSI amplitude data are extracted from the WIFI signal, SSA may be first used for denoising, and then HHT may be used to obtain a spectrum diagram. Finally, the CSI amplitude data of fall or fall-like may be extracted and used as training or testing data of the deep neural network. Herein, since the CSI amplitude data to be identified which are input into the deep neural network are data of possible occurrence of fall or fall-like, the deep neural network may be used to distinguish falls in fine-grained level, so as to distinguish between fall and fall-like better.

In an exemplary embodiment, the deep neural network may include: a Deep Convolutional Neural Network (DCNN), a Long Short-Term Memory neural network (LSTM), and a classifier, wherein, output data of the DCNN are input to the LSTM, and output data of the LSTM are input to the classifier. Herein, the DCNN has the ability of feature extraction and transtformation, and the LSTM has the ability to distinguish similar activities, for example, it may distinguish falls in a fine-grained level, such as identifying a fall-like behavior.

In an exemplary embodiment, the DCNN may include three convolution layers, three pooling layers, and a full connection layer. The first convolutional layer connects the first pooling layer, the first pooling layer connects the second convolutional layer, the second convolutional layer connects the second pooling layer, the second pooling layer connects the third convolutional layer, the third convolutional layer connects the third pooling layer, and the third pooling layer connects the full connection layer.

In an exemplary embodiment, the number of neurons in the LSTM may be 30 and the hyperbolic tangent function tan h is used as the activation function of the output and memory units.

In an exemplary embodiment, the classifier may include a SOFTMAX classifier. However, this application is not limited thereto. In other implementations, other types of classifiers may be used.

In this embodiment, by combining the DCNN and LSTM and using the SOFTMAX classifier, the final fall detection result is obtained, thereby improving the detection accuracy.

In an exemplary embodiment, before step 101, the fall detection method of this embodiment may also include: extracting the CSI data from the WIFI signal received in the detection area, preprocessing the CSI data to obtain CSI data of fall and fall-like; and using the CSI data of fall and fall-like to train the deep neural network.

In this exemplary embodiment, the process of steps 101 and 102 may be referred to to obtain the training data and train the deep neural network, so that the deep neural network may be used for distinguishing between fall and fall-like in the detection area or similar environments.

FIG. 2 is a schematic diagram of a fall detection device provided by an embodiment of this application. As shown in the FIG. 2, the fall detection device provided by the present embodiment includes a receiving module 201, a preprocessing module 202 and a deep neural network 203.

The receiving module 201 is adapted to receive a WIFI signal transmitted by a transmitter in a detection area, and extract CSI data from the WIFI signal. The preprocessing module 202 is adapted to preprocess the CSI data to obtain CSI data to be identified. The deep neural network 203 is adapted to process the CSI data to be identified to determine whether a target object falls in the detection area.

In an exemplary embodiment, the receiving module 201 may include a receiving antenna, which is adapted to receive a WIFI signal in the detection area.

In an exemplary embodiment, the CSI data may include CSI amplitude data. The preprocessing module 202 may perform preprocessing on the CSI data in the following way to obtain the CSI data to be identified: using an SSA algorithm to denoise the CSI amplitude data; converting the denoised CSI amplitude data into a spectrum diagram by HHT; and extracting CSI amplitude data of fall or fall-like from the spectrum graph to be used as the CSI data to be identified.

In an exemplary embodiment, the deep neural network 203 may include: a DCNN, an LSTM, and a classifier (for example, SOFTMAX classifier).

The relevant description of the fall detection device provided in this embodiment may refer to the relevant description of the fall detection method mentioned above, which will not be repeated here.

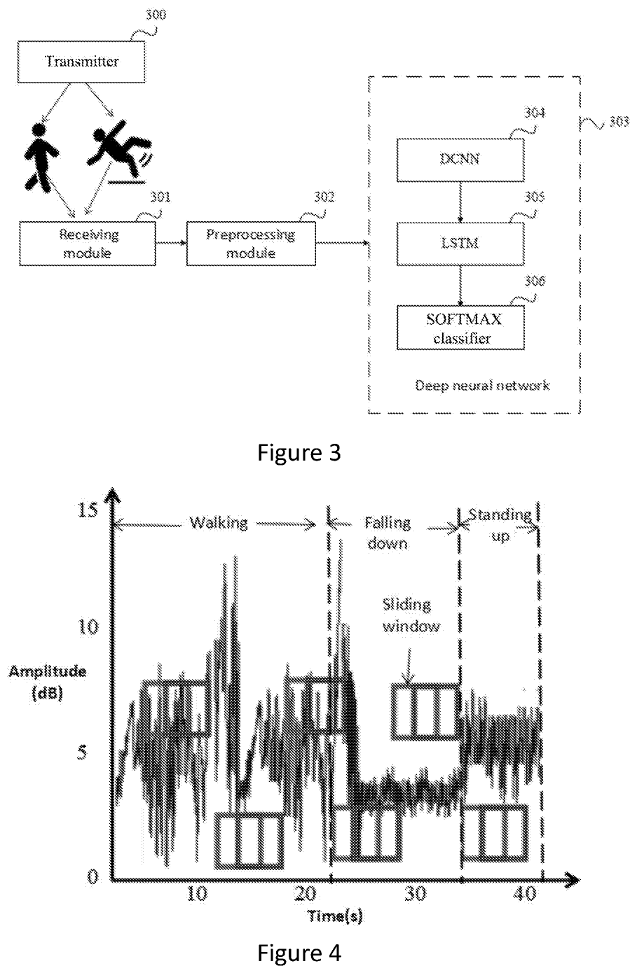

FIG. 3 is a schematic diagram of an application example provided by an embodiment of the present application. In this embodiment, the detection of whether a user (a target object) falls in a bathroom (a detection area) is illustrated as an example. In this embodiment, a transmitter (for example, a transmitter 300) and a data processing terminal may be configured in the detection area. Herein, the transmitter 300 is adapted to transmit a WIFI signal to the detection area; the data processing terminal is adapted to receive the WIFI signal, and conduct fall detection and processing based on the WIFI signal. However, this application is not limited thereto. In other implementations, at least two transmitters may be configured in the detection area to improve the coverage of the WIFI signal. In addition, since the WIFI signal can penetrate a wall, the data processing terminal capable of receiving a WIFI signal may be configured within or outside the detection area.

As shown in FIG. 3, the data processing terminal (such as the fall detection device shown in FIG. 2) may include a receiving module 301, a preprocessing module 302, and a deep neural network 303. The deep neural network 303 may include a DCNN 304, an LSTM 305 and a SOFTMAX classifier 306.

In this embodiment, the receiving module 301 may include a receiving antenna, which is adapted to receive a WIFI signal. Moreover, after the receiving module 301 receives the WIFI signal, CSI amplitude data may be extracted from the WIFI signal and transmitted to the preprocessing module 302. For example, after the receiving module 301 receives the WIFI signal, CSI raw data may be extracted from the WIFI signal firstly, and then CSI amplitude data may be extracted through analysis.

In this embodiment, after receiving CSI amplitude data, the preprocessing module 302 uses the SSA algorithm for denoising first, and then uses HHT for conversion into a spectrum diagram, and finally extracts the data of fall and fall-like from the spectrum diagram to be used as training or testing data of the deep neural network 303.

SSA algorithm is divided into the following two stages: decomposition and reconstruction. In the first stage, numbers are arranged in a trajectory matrix by means of embedding, and then a singular spectrum is obtained by decomposing the matrix through singular values. In the second stage, a rank of the trajectory matrix is reduced, and then a signal after noise attenuation is reconstructed according to the trajectory matrix with a reduced rank.

In this embodiment, a spectrum diagram for positioning time and frequency may be obtained through HHT. The HHT in this embodiment may include the following two parts: Empirical Mode Decomposition (EMD) and Hilbert Spectrum Analysis (HSA). A general process of HHT signal processing by HHT is to firstly use EMD to decompose a given signal into several Intrinsic Mode Functions (IMFs), and the IMFs are components that satisfy conditions. Then, Hilbert transform is performed on each IMF to obtain a corresponding Hilbert spectrum, that is, each IMF is represented in a joint time domain. Finally, the Hilbert spectrum of an original signal is obtained by summarizing all Hilbert spectra of all the IMFs. Compared with traditional Fourier transform and wavelet transform, HHT has the following significant advantages: it can analyze non-linear and non-stationary signals, has complete adaptability, and is suitable for abrupt signals, and an instantaneous frequency is obtained by derivation.

In this embodiment, since different human activities occupy different spectral bands, the spectral classification of each window can be analyzed for activity classification. In this embodiment, an adaptive sliding window is used to divide two different types of human activities (fall and non-fall). It should be noted that only data of fall or fall-like are extracted from the spectrum, and data of obvious non-fall are not extracted.

A process of extracting CSI amplitude data of fall or fall-like from a spectrum diagram is illustrated below with reference to FIG. 4.

In general, frequencies of a range between 3 Hz and 25 Hz may be divided. In this embodiment, low frequency (fL) is defined as 3 to 10 Hz and high frequency (fH) as 10 to 25 Hz. In addition, any frequency below 0.2 Hz will be removed as noise. Typically, on-the-ground activities (such as lying on the ground) may include IL, while off-the-ground activities (such as sitting or standing) may include fL and fH. Based on this, a fall event first occupies a higher spectral band corresponding to rapid movement, and then occupies a lower spectral band corresponding to lying. For example, if a previous window w1 contains fL and fH, and a second window w2 contains fL, then a window w3 may be obtained through a combination of the window w1 and the window w2, the window w3 may correspond to an activity of fall or fall-like. Therefore, window w3 may be selected to be subsequently input into the deep neural network for feature extraction and classification.

In this embodiment, the LSTM and DCNN are combined to form an LSTM-DCNN network model. the LSTM is good at sequence structure analysis and the DCNN is good at feature extraction and transformation. The output of the LSTM-DCNN network model at each moment is provided to the SOFTMAX classifier for probability calculation, so as to get the final result of whether fall occurs. The SOFTMAX classifier may use a cost function in a cross entropy form to calculate the decision result.

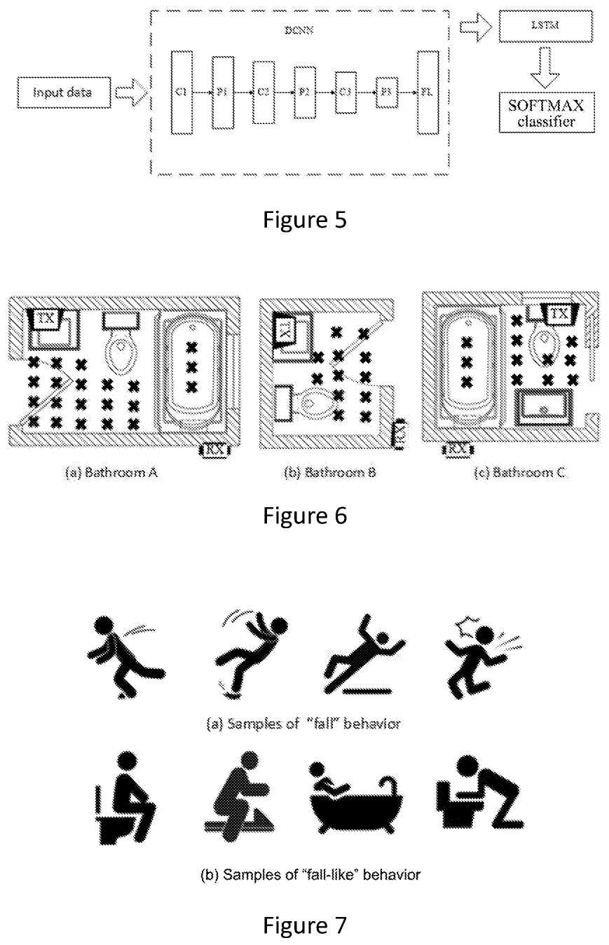

FIG. 5 is a schematic diagram of the construction of a deep neural network in an embodiment of this application. As shown in FIG. 5, a size of input data of the DCNN is 128*128, and a pixel value is between 0 and 255. The DCNN may include three convolution layers (e.g., C1, C2, C3), three pooling layers (e.g., P1, P2, P3), and a full connection layer (FL). The first convolution layer C1 may include 64 feature mappings, the second convolution layer C2 and the third convolution layer C3 may include 128 and 256 feature mappings respectively. As shown in FIG. 5, the output of the first layer convolution C1 is provided to the first pooling layer P1, the output of the first pooling layer P1 is provided to the second convolution layer C2, the output of the second layer convolution C2 is provided to the second pooling layer P2, the output of the second pooling layer P2 is provided to the third convolution C3, the output is of the third layer convolution C3 is provided to the third layer pooling P3, and the output of the third layer pooling P3 is provided to the full connection layer FL. The number of neurons of the LSTM may be 30, and the hyperbolic tangent function tan h is used as the activation function of output and memory units. The SOFTMAX classifier may contain two neurons. In this embodiment, updating of network parameters in the deep neural network may use the combination of batch training and adaptive gradient adjustment.

FIG. 6 is a schematic diagram of three data collection environments in an embodiment of this application. As human behavior identification by using WIFI signals is greatly affected by different environments, in this embodiment, training for a deep neural network may be conducted based on data collected in three different bathroom environments to improve the detection performance of the deep neural network. As shown in FIG. 6, for three different bathroom environments, a transmitter (TX) (for example, a router) is placed in each bathroom, and a data processing terminal (for example, a laptop that includes a WIFI receiver (RX) of which a sampling rate may be 1 KHz) is placed outside the bathroom. In this embodiment, in order to make the WIFI signal coverage wider, the transmitter and receiver may be placed in the diagonal direction of the bathroom, that is, at both ends of the diagonal line of the bathroom. In FIG. 6, the cross symbol represents a position where a fall or non-fall behavior occurs in the bathroom.

In the process of fall detection, a fall-like in the bathroom (for example, squatting in the toilet or lying in the bathtub in the bathroom) may be mistaken as a fall. Therefore, in this embodiment, after collecting CSI amplitude data, CSI amplitude data of fall and fall-like are extracted and used as training data to be input into the deep neural network, so as to train the deep neural network to distinguish between fall and fall-like.

FIG. 7 is an exemplary diagram of fall and fall-like in this embodiment. Fall behaviors in this embodiment are divided into static fall and motile fall. Static fall may refer to fall from a stationary position, such as fall while sitting or standing. Motile fall may refer to fall or trip while walking, including fall forward, fall backward or fall sideways. A fall-like is a behavior similar to a fall, but not an actual fall. For example, it may include sitting, walking and then sitting, walking and then lying down, and standing and then lying down. As shown in the FIG. 7, a fall-like behavior of a user in the bathroom may include squatting in the toilet, squatting on the closestool, bathing, lying on the closestool and so on.

In this embodiment, multiple experiments of fall and fall-like may be conducted in the bathroom A, bathroom B and bathroom C as shown in FIG. 6. In this way, multiple sets of data of fall and fall-like in different bathrooms may be collected to train the deep neural network, so as to improve the accuracy of fall detection by the deep neural network in different bathroom scenarios.

In this embodiment, CSI amplitude data extracted from a WIFI signal are converted into a spectrum diagram, and the DCNN and LSTM are combined to extract features of the CSI amplitude data, and SOFTMAX classifier is used for final classification and recognition, so as to detect whether the target object falls in the detection area. Herein, the LSTM can automatically extract features, data preprocessing is not even needed, and the LSTM can maintain temporal state information of activities, that is, LSTM has the potential to distinguish similar activities, such as the distinguishing between “lying down” and “falling down”. In this way, fall behaviors may be distinguished in a fine-grained level, for example, the behavior of “lying in the bathtub” may not be mistaken as a behavior of fall.

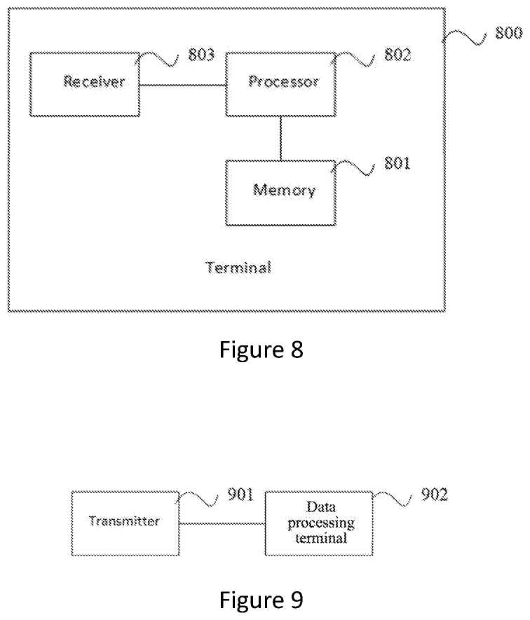

FIG. 8 is a schematic diagram of a terminal provided by an embodiment of this application. As shown in the FIG. 8, a terminal 800 provided in the embodiment of this application includes a receiver 803, a memory 801 and a processor 802. The receiver 803 is connected with the processor 802, and is adapted to receive a WIFI signal in a detection area. The memory 801 is adapted to store a fall detection program, which, when executed by the processor 802, implements the steps of the fall detection method provided by the above embodiment, such as the steps shown in FIG. 1. Those skilled in the art could understand that the structure shown in the FIG. 8 is only a schematic diagram of partial structure related to the solution of this application and does not constitute the limit on the terminal 800 to which the solution of this application is applied. The terminal 800 may contain more or fewer parts than shown in the figure, or combine some parts, or have different layouts of parts.

The processor 802 may include, but not limited to, a processing device such as a Microcontroller Unit (MCU) or Field Programmable Gate Array (FPGA). Memory 801 may be used for storing software programs and modules of applications, such as program instructions or modules corresponding to the fall detection method. The processor 802 implements various functional applications and data processing, such as implementing the fall detection method provided by the embodiment, by running the software programs and modules stored in the memory 801. The memory 801 may include high-speed random-access memory as well as non-volatile memory, such as one or more magnetic storage devices, flash memories, or other non-volatile solid-state memories. In some examples, the memory 801 may include a memory set remotely from the processor 802, and the remote memory may be connected to the terminal 800 through a network. An example of such a network includes but not limited to the Internet, enterprise intranet, local area network, mobile communication network, and a combination thereof.

In addition, the description of the relevant implementation process of the terminal provided by this embodiment may refer to the related description of the fall detection method and the fall detection device mentioned above, so it is not repeated here.

FIG. 9 is a schematic diagram of a fall detection system provided by an embodiment of this application. As shown in the FIG. 9, the fall detection system provided by the present embodiment is used to detect a status of a target object in a detection area, and includes a transmitter 901 and a data processing terminal 902.

The transmitter 901 may be adapted to transmit a WIFI signal in the detection area. The data processing terminal 902 may be adapted to receive the WIFI signal transmitted by the transmitter 901 in the detection area and extract CSI data from the WIFI signal. The CSI data are preprocessed to obtain CSI data to be identified. The CSI data to be identified are processed by a deep neural network to determine whether the target object falls in the detection area.

In addition, the description of the relevant implementation process of the fall detection system provided by this embodiment may refer to the related description of the fall detection method and the fall detection device mentioned above, so it is not repeated here.

In addition, an embodiment of the application also provides a computer-readable medium in which a fall detection program is stored. When the fall detection program is executed by a processor, steps of the fall detection method provided by the above embodiment, for example, steps as shown in the FIG. 1, are implemented.

Those of ordinary skill in the art may understand that all or some of the steps, systems, and functional modules/units in the methods disclosed above may be implemented as software, firmware, hardware, and their appropriate combinations. In the hardware embodiment, the division between functional modules/units mentioned in the above description does not necessarily correspond to the division of physical components. For example, a physical component may have multiple functions, or a function or step may be performed by several physical components working together. Some or all of the components may be implemented as software executed by processors, such as digital signal processors or microprocessors, or as hardware, or as integrated circuits, such as application-specific integrated circuits. Such software may be distributed on computer readable media, which may include computer storage media (or non-temporary media) and communication media (or temporary media). As well known to those of ordinary skill in the art, the term computer storage media includes transitory, and non-transitory, removable, and non-removable media implemented in any method or technology used for storing information (such as computer readable instructions, data structures, program modules or other data). Computer storage media include, but not limited to, RAM, ROM, EEPROM, flash memory or other storage technology, CD-ROM, Digital Video Disk (DVD) or other optical disk storage, magnetic box, magnetic tape, disk storage or other magnetic storage device, or any other media that may be used to store desired information and may be accessed by the computer. In addition, it is well known to those of ordinary skill in the art that the communication media typically include computer-readable instructions, data structures, program modules, or other data in modulated data signals such as carriers or other transmission mechanisms, and may include any information transmission medium.

What is claimed is:

1. A fall detection method for detecting whether a target object falls in a detection area, comprising:
receiving a WIFI signal transmitted by a transmitter in the detection area, and extracting channel state information (CSI) data from the WIFI signal;
preprocessing the CSI data to obtain CSI data to be identified; and
processing the CSI data to be identified through a deep neural network to determine whether the target object falls in the detection area.

2. The method according to claim 1, wherein the CSI data comprises CSI amplitude data.

3. The method according to claim 2, wherein preprocessing the CSI data to obtain the CSI data to be identified comprises:
denoising the CSI amplitude data by using a Singular Spectrum Analysis (SSA) algorithm;
converting the denoised CSI amplitude data into a spectrum diagram by Hilbert-Huang Transform (HHT); and
extracting CSI amplitude data of fall or fall-like from the spectrum diagram to be used as the CSI data to be identified.

4. The method according to claim 1, wherein the deep neural network comprises: a deep convolutional neural network (DCNN), a long short-term memory neural network (LSTM), and a classifier, wherein, output data of the DCNN is input into the LSTM, and output data of the LSTM is input into the classifier.

5. The method according to claim 4, wherein the DCNN comprises three convolution layers, three pooling layers and a full connection layer.

6. The method according to claim 4, wherein the number of neurons in the LSTM is 30, and a hyperbolic tangent function tan h is used as an activation function of output and memory units.

7. The method according to claim 4, wherein the classifier comprises a SOFTMAX classifier.

8. The method according to claim 1, wherein the method also comprises:
extracting CSI data from the WIFI signal received in the detection area; preprocessing the CSI data to obtain CSI data of fall and fall-like; and training the deep neural network by using the CSI data of fall and fall-like.

9. A fall detection device for detecting whether a target object falls in a detection area, comprising:
a receiving module adapted to receive a WIFI signal transmitted by a transmitter in the detection area, and extract channel state information (CSI) data from the WIFI signal;
a preprocessing module adapted to preprocess the CSI data to obtain CSI data to be identified; and
a deep neural network adapted to process the CSI data to be identified to determine whether the target object falls in the detection area.

10. A terminal comprising a receiver, a memory, and a processor, wherein the receiver is connected to the processor and is adapted to receive a WIFI signal transmitted by a transmitter in a detection area; the memory is adapted to store a fall detection program executable by the processor to implement the steps of the fall detection method as claimed in claim 1.

11. A fall detection system for detecting whether a target object falls in a detection area, comprising a transmitter and a data processing terminal;
wherein the transmitter is adapted to transmit a WIFI signal in the detection area;
the data processing terminal is adapted to receive the WIFI signal transmitted by the transmitter in the detection area, and extract channel state information (CSI) data from the WIFI signal; preprocess the CSI data to obtain CSI data to be identified; and process the CSI data to be identified through a deep neural network to determine whether the target object falls in the detection area.

12. A computer-readable medium, in which a fall detection program is stored for implementing steps of the fall detection method as claimed in claim 1 when the fall detection program is executed by a processor.
